(19) United States
(12) Patent Application Publication
(10) Pub. No.: US 2021/0177637 A1
(43) Pub. Date: Jun. 17, 2021

(54) POSTURE CORRECTION BRACE

(71) Applicant: Delco Innovations LLC, Bethlehem, PA (US)

(72) Inventors: Blaise Derrico, Breinigsville, PA (US); Brandon Boyer, Breinigsville, PA (US)

(73) Assignee: Delco Innovations LLC, Bethlehem, PA (US)

(21) Appl. No.: 17/084,036

(22) Filed: Oct. 29, 2020

Related U.S. Application Data
(60) Provisional application No. 62/947,364, filed on Dec. 12, 2019.

(51) Int. Cl. A61F 5/02

(52) U.S. Cl. CPC A61F 5/026; A61F 5/028

(57) ABSTRACT

One aspect of the invention provides a back brace including: a first shoulder strap and a second shoulder strap, wherein each shoulder strap comprises at least a dorsal end; a waist belt configured to extend circumferentially around an object; and a pulley system. The pulley system includes: a set of thoracic pulley wheels coupled to the dorsal ends of the first shoulder strap and the second shoulder strap; a set of lumbar pulley wheels coupled to a center portion of the waist belt; and at least one cord looped around the set of lumbar pulley wheels and the set of thoracic pulley wheels. The thoracic pulley wheels travel proximally towards the set of lumbar pulley wheels when a free end of the cord is pulled.

### CROSS-REFERENCE TO RELATED APPLICATION

This application claims the benefit of priority under 35 U.S.C. § 119(e) of U.S. Provisional Patent Application Ser. No. 62/947,364, filed Dec. 12, 2019. The entire content of this application is hereby incorporated by reference herein.

### BACKGROUND OF THE INVENTION

Back braces can be used to alter the positioning of back-associated body parts of an individual.

### SUMMARY OF THE INVENTION

One aspect of the invention provides a back brace including: a first shoulder strap and a second shoulder strap, wherein each shoulder strap comprises at least a dorsal end; a waist belt configured to extend circumferentially around an object; and a pulley system. The pulley system includes: a set of thoracic pulley wheels coupled to the dorsal ends of the first shoulder strap and the second shoulder strap; a set of lumbar pulley wheels coupled to a center portion of the waist belt; and at least one cord looped around the set of lumbar pulley wheels and the set of thoracic pulley wheels. The thoracic pulley wheels travel proximally towards the set of lumbar pulley wheels when a free end of the cord is pulled.

This aspect of the invention can have a variety of embodiments. The back brace can further include a handle coupled to the free end of the cord.

The back brace can further include: a first portion of a hook-and-loop system coupled to an exterior surface of the waist belt and a second portion of the hook-and-loop system coupled to an interior surface of the waist belt.

The set of lumbar pulley wheels and the set of thoracic pulley wheels can each include a first pulley system and a second pulley system. The first pulley system can be coupled to the dorsal end of the first shoulder strap and the second pulley system can be coupled to the dorsal end of the second shoulder strap.

Another aspect of the invention provides a back brace consisting of: a first shoulder strap and a second shoulder strap, wherein each shoulder strap comprises at least a dorsal end; a waist belt configured to extend circumferentially around an object; one or more pulley wheels coupled to a center portion of the waist belt; and one or more cords. The one or more cords are: coupled to the dorsal ends of the first shoulder strap and the second shoulder strap; and looped around the one or more pulley wheels. The first shoulder strap and the second shoulder strap travel towards the one or more pulley wheels when a free end of the one or more cords is pulled.

### BRIEF DESCRIPTION OF THE DRAWINGS

For a fuller understanding of the nature and desired objects of the present invention, reference is made to the following detailed description taken in conjunction with the accompanying drawing figures, wherein like reference characters denote corresponding parts throughout the several views.

FIGS. 1 and 2 depict posterior views of a posture correction brace according to embodiments of the claimed invention.

FIGS. 3 and 4 depict anterior views of a posture correction brace according to embodiments of the claimed invention.

FIGS. 5 and 6 depict posterior views of a posture correction brace according to embodiments of the claimed invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

### Posture Correction Brace

The disclosure described herein relates to a posture correction brace. The brace can include a waist belt, shoulder straps, and a pulley system. By adjusting the pulley system (e.g., tightening tension or releasing tension on the pulley system), the ends of the pulley system can be drawn towards or further away from each other. As the ends of the pulley system can be attached to the waist belt and the shoulder straps, the length of the brace can be altered. Altering the length of the brace can also alter the posture of the wearer. Further, as the belt does not include a rigid frame, the brace provides a more comfortable and effective means of posture correction as opposed to conventional back braces.

### Waist Belt

The posture correction brace can include a waist belt. The waist belt can include a piece of fabric or other flexible material (e.g., polyester, etc.). The waist belt can wrap around the waist of a wearer. Free ends of the waist belt can attach to one another to attach the waist belt to the wearer (e.g., via clips, strings, buckles, belt buckle and notches, a hook-and-loop system, and the like), and, in some cases, modify the fitting of the waist belt around the wearer (e.g., tightening, loosening the waist belt, etc.).

### Shoulder Straps

The posture correction brace can include a pair of shoulder straps. The shoulder straps can be made of fabric or other flexible material. Each shoulder strap can include a first end (e.g., a lumbar end) and a second end (e.g., a dorsal end). The first end can couple to the waist belt. The second end can couple to a top end of the pulley system described below. In some cases, the shoulder straps can also include a pair of horizontal couplers (e.g., sternum straps), that can couple to one another. The horizontal couplers, when coupled together, and pull the shoulder straps towards one another when worn by a wearer.

### Pulley System

The posture correction brace can include a pulley system. The pulley system can include at least two axles. A first (e.g., thoracic) axle can be attached (e.g., via stitching, fusing, stapling, and the like) to the second ends of the shoulder straps. A second (e.g., lumbar) axle can be attached to the waist belt (e.g. in a lumbar region of the waist belt). Thus, the overall length of the pulley system spans from the waist belt to the second ends of the shoulder straps. At least one cord can be threaded through the pulley system (e.g., via the axles). At least one end of the cord can be moveable (e.g., uncoupled to the brace). By applying force to the free end(s) of the cord, a wearer can apply tension to the pulley system. The axles of the pulley system can be drawn towards one another from the applied tension. As the positioning of the waist belt remains relatively static, a wearer of the posture correction brace can experience a force on the upper back due to the drawing of the axles together, pulling the shoulder blades of the wearer down and back towards the lumbar region. As the pulley system relies on lengths of cord to achieve the desired force, a wearer does not experience discomfort (e.g., due to a rigid external frame) in wearing and applying force to the posture correction brace. Additionally, the cord may be selected to have a desired amount of stretch to accommodate movement.

### Exemplary Embodiment

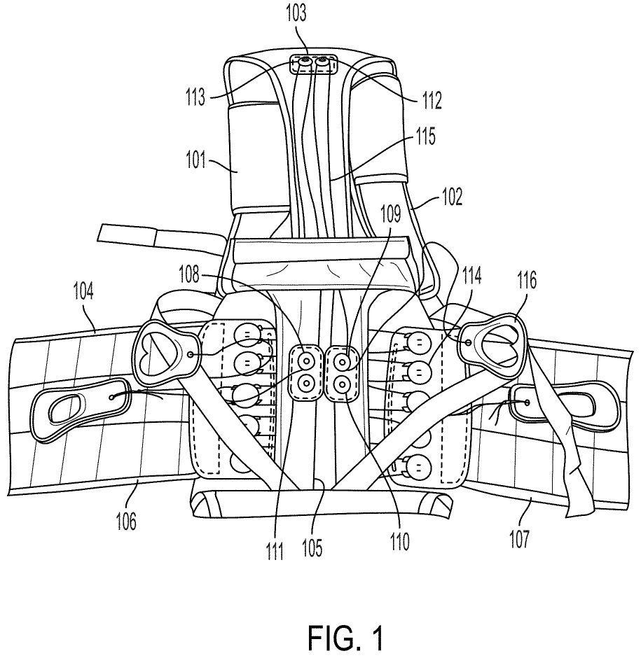

FIG. 1 depicts an exemplary embodiment of the posture correction brace according to the claimed invention. The posture correction brace can include first and second shoulder straps 101 and 102, respectively. Each shoulder strap includes a first end and a second end. The first end of the shoulder straps can couple to on another, for example at point 103 of the posture correction brace. However, in some embodiments the first ends can couple to one another at various points on the brace, and in some cases stay isolated from one another (e.g., for a brace with separate pulley systems coupled to each of the shoulder straps).

Likewise, the second ends of the shoulder straps can couple to the waist belt 104 at point 105. A first portion of the shoulder straps can be composed of fabric (e.g., polyester, nylon, etc.) for increased comfort for a wearer. A second portion of the shoulder strap can be composed of threading or cording, which can provide for increased flexibility and/or facilitate adjustability of the shoulder strap.

The waist belt 104 can wrap around the waist of the wearer. The waist belt 104 can be composed of fabric material for comfort, adjustability, etc. The waist belt can include a hook-and-loop fastening system (e.g., Velcro®) on the surfaces of the waist belt 104. For example, an array of hooks can be attached to an inner surface of one end of the waist belt 104, and an array of loops can be attached to an outer surface of an opposite end of the waist belt, or vice versa. When the ends of the waist belt 104 (e.g., ends 106 and 107) overlap one another, the hooks of the inner surface can couple to the loops of the outer surface.

The posture correction brace can also include at least one pulley system. In the embodiment illustrated in FIG. 1, the posture correction brace includes two pulley systems: pulley systems 108 and 109. The pulley systems 108 and 109 can include top axles and bottom axles. The top axles 112 and 113 can couple to the respective shoulder strap or straps at the point 103 (e.g., through stitching, rivets, adhesive, and like). The bottom axles 110 and 111 can couple to the waist belt 104 in the lumbar region. Thus, a total length of the pulley systems 108 and 109 can span a total spine length of the posture correction brace. Further, periphery axles 114 can also be a part of the pulley systems 108 and/or 109. The periphery axles 114 can increase the number of axles included within the pulley systems, thereby reducing the required force for adjusting the posture correction brace.

Cording 115 can be wound through the axles of the respective pulley systems. The cording can include a working load and/or thickness characteristics sufficient to operate within the pulleys systems. An end of the cording 115 can be coupled to a bottom axle, such as bottom axle 110. The other end of the cording 115 can be wound through the pulley system 109 (e.g., top axle 112 and periphery axles 114). The other end can then be coupled to a handle 116. The handle 116 can provide a wearer with an easier ability to grip the cording end as opposed to the cording end itself. The handle 116 can be designed in any shape and composed of any material known in the relevant art. The handle 116 can also include a hook or loop array on an interior surface, such that when the handle 116 is pulled away from the bottom axle, the handle 116 can be coupled to the exterior surface of the waist belt 104 (e.g., to maintain the desired, applied tension).

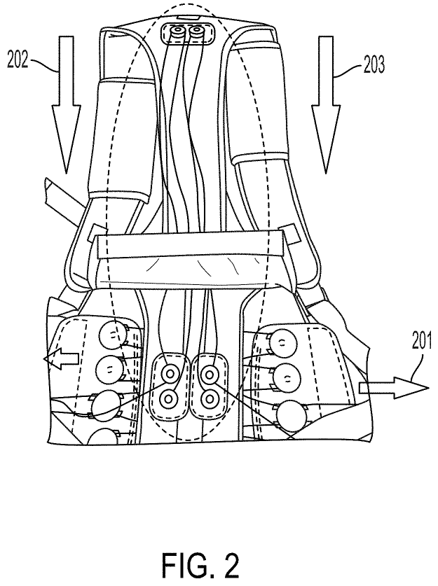

FIG. 2 depicts a posture correction brace, along with arrows illustrating the direction of force applied when tension is increased on the pulley systems. For example, direction arrow 201 illustrates a distal direction of force that is applied when a wearer pulls the cord end or handle of a pulley system. The cord end or handle will extend distally away from the pulley system. In turn, the direction arrows 202 and 203 illustrate the downward force that is applied to the pulley system from the wearer pulling the cord end or handle. This downward force will pull the top portion of the posture correction brace (e.g., through force applied at the top axles) towards for the bottom of the posture correction brace (e.g., towards the bottom axles).

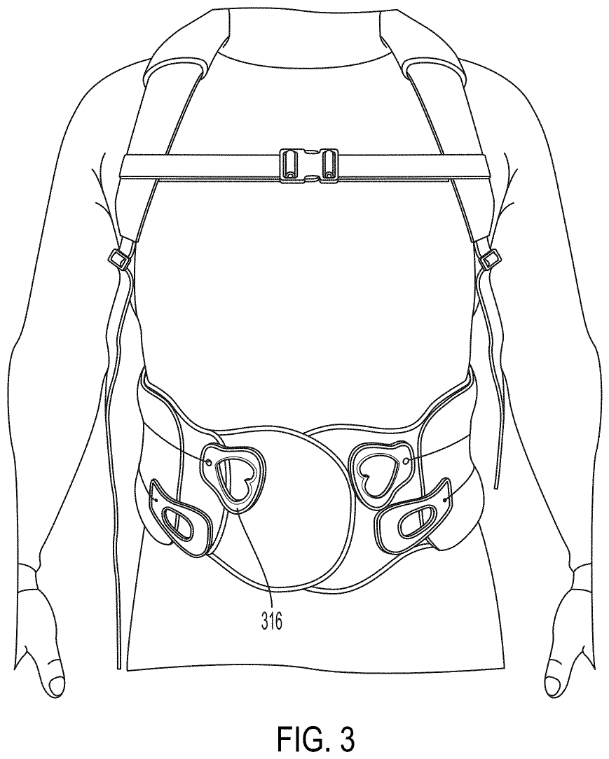

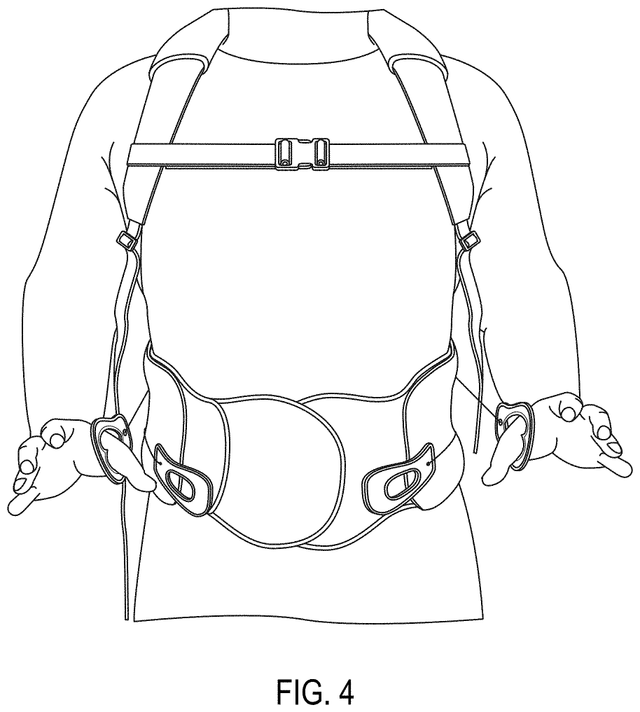

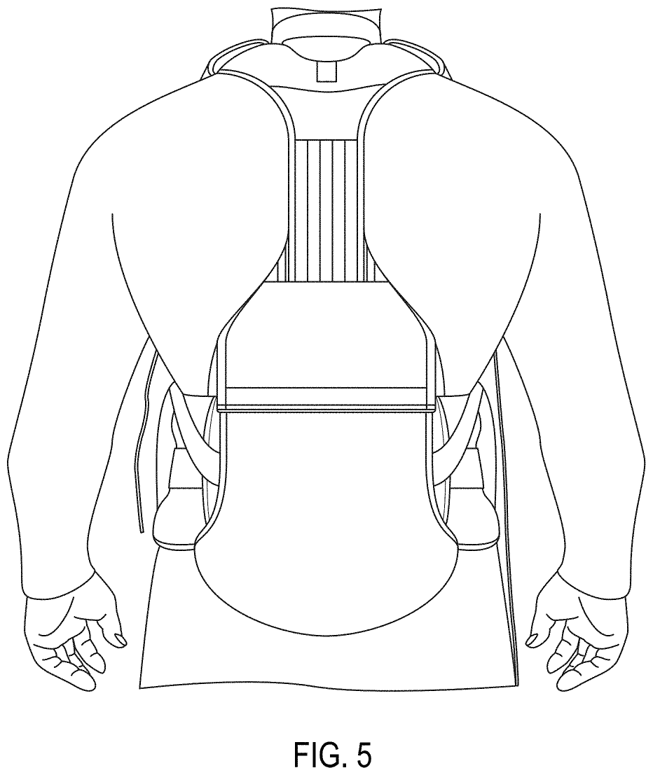

FIG. 3 depicts a posture correction brace that is worn by the wearer. The handles 316 are connected to the waist belt through the hook and loop system described in FIG. 1. FIG. 4 depicts a wearer in the process of pulling the handles for adjusting the tension applied to the posture correction brace. FIG. 5 depicts a posterior perspective of a posture correction brace. A fabric cover is over the pulley system, which can protect the pulley system from environmental factors and/or for aesthetic purposes.

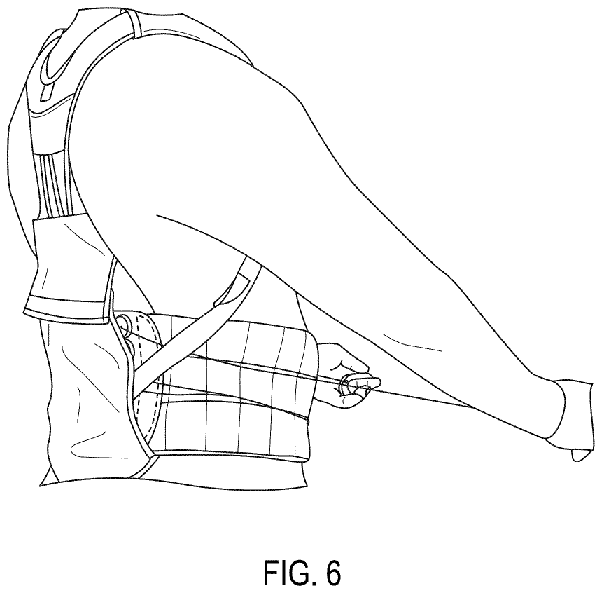

FIG. 6 depicts a posterior perspective of a posture correction brace worn by a wearer, where the wearer is in the process of applying tension to the pulley system(s) of the posture correction brace.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A back brace, comprising:
a first shoulder strap and a second shoulder strap, wherein each shoulder strap comprises at least a dorsal end;
a waist belt configured to extend circumferentially around an object; and
a pulley system, comprising:
a set of thoracic pulley wheels coupled to the dorsal ends of the first shoulder strap and the second shoulder strap;
a set of lumbar pulley wheels coupled to a center portion of the waist belt; and
at least one cord looped around the set of lumbar pulley wheels and the set of thoracic pulley wheels, wherein the thoracic pulley wheels travel proximally towards the set of lumbar pulley wheels when a free end of the cord is pulled.

2. The back brace of claim 1 further comprising:
a handle coupled to the free end of the cord.

3. The back brace of claim 1, further comprising:
a first portion of a hook-and-loop system coupled to an exterior surface of the waist belt; and
a second portion of the hook-and-loop system coupled to an interior surface of the waist belt.

4. The back brace of claim 1, wherein the set of lumbar pulley wheels and the set of thoracic pulley wheels each comprise a first pulley system and a second pulley system, wherein the first pulley system is coupled to the dorsal end of the first shoulder strap and the second pulley system is coupled to the dorsal end of the second shoulder strap.

5. A back brace consisting of:
a first shoulder strap and a second shoulder strap, wherein each shoulder strap comprises at least a dorsal end;
a waist belt configured to extend circumferentially around an object;
one or more pulley wheels coupled to a center portion of the waist belt; and
one or more cords:
coupled to the dorsal ends of the first shoulder strap and the second shoulder strap; and
looped around the one or more pulley wheels;
wherein the first shoulder strap and the second shoulder strap travel towards the one or more pulley wheels when a free end of the one or more cords is pulled.
